SEQUENCE LISTING (9 sequences)

SEQ ID NO: 1 - DNA, 1896 bp, Homo sapiens
  CDS (1)..(1893)
atggtttctaaactgagccagctgcagacggagctcctggcggccctgctcgagtcagggctgagcaaagaggcactgatccaggcactgggtgagccggggccctacctcctggctggagaaggccccctggacaagggggagtcctgcggcggcggtcgaggggagctggctgagctgcccaatgggctgggggagactcggggctccgaggacgagacggacgacgatggggaagacttcacgccacccatcctcaaagagctggagaacctcagccctgaggaggcggcccaccagaaagccgtggtggagacccttctgcaggaggacccgtggcgtgtggcgaagatggtcaagtcctacctgcagcagcacaacatcccacagcgggaggtggtcgataccactggcctcaaccagtcccacctgtcccaacacctcaacaagggcactcccatgaagacgcagaagcgggccgccctgtacacctggtacgtccgcaagcagcgagaggtggcgcagcagttcacccatgcagggcagggagggctgattgaagagcccacaggtgatgagctaccaaccaagaaggggcggaggaaccgtttcaagtggggcccagcatcccagcagatcctgttccaggcctatgagaggcagaagaaccctagcaaggaggagcgagagacgctagtggaggagtgcaatagggcggaatgcatccagagaggggtgtccccatcacaggcacaggggctgggctccaacctcgtcacggaggtgcgtgtctacaactggtttgccaaccggcgcaaagaagaagccttccggcacaagctggccatggacacgtacagcgggccccccccagggccaggcccgggacctgcgctgcccgctcacagctcccctggcctgcctccacctgccctctcccccagtaaggtccacggtgtgcgctatggacagcctgcgaccagtgagactgcagaagtaccctcaagcagcggcggtcccttagtgacagtgtctacacccctccaccaagtgtcccccacgggcctggagcccagccacagcctgctgagtacagaagccaagctggtctcagcagctgggggccccctcccccctgtcagcaccctgacagcactgcacagcttggagcagacatccccaggcctcaaccagcagccccagaacctcatcatggcctcacttcctggggtcatgaccatcgggcctggtgagcctgcctccctgggtcctacgttcaccaacacaggtgcctccaccctggtcatcggcctggcctccacgcaggcacagagtgtgccggtcatcaacagcatgggcagcagcctgaccaccctgcagcccgtccagttctcccagccgctgcacccctcctaccagcagccgctcatgccacctgtgcagagccatgtgacccagagccccttcatggccaccatggctcagctgcagagcccccacgccctctacagccacaagcccgaggtggcccagtacacccacacgggcctgctcccgcagactatgctcatcaccgacaccaccaacctgagcgccctggccagcctcacgcccaccaagcaggtcttcacctcagacactgaggcctccagtgagtccgggcttcacacgccggcatctcaggccaccaccctccacgtccccagccaggaccctgccggcatccagcacctgcagccggcccaccggctcagcgccagccccacagtgtcctccagcagcctggtgctgtaccagagctcagactccagcaatggccagagccacctgctgccatccaaccacagcgtcatcgagaccttcatctccacccagatggcctcttcctcccagtaa

SEQ ID NO: 2 - protein, 631 aa, Homo sapiens
MVSKLSQLQTELLAALLESGLSKEALIQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMDTYSGPPPGPGPGPALPAHSSPGLPPPALSPSKVHGVRYGQPATSETAEVPSSSGGPLVTVSTPLHQVSPTGLEPSHSLLSTEAKLVSAAGGPLPPVSTLTALHSLEQTSPGLNQQPQNLIMASLPGVMTIGPGEPASLGPTFTNTGASTLVIGLASTQAQSVPVINSMGSSLTTLQPVQFSQPLHPSYQQPLMPPVQSHVTQSPFMATMAQLQSPHALYSHKPEVAQYTHTGLLPQTMLITDTTNLSALASLTPTKQVFTSDTEASSESGLHTPASQATTLHVPSQDPAGIQHLQPAHRLSASPTVSSSSLVLYQSSDSSNGQSHLLPSNHSVIETFISTQMASSSQ

SEQ ID NO: 3 - DNA, 1425 bp, Homo sapiens
  CDS (1)..(1422)
atgcgactctccaaaaccctcgtcgacatggacatggccgactacagtgctgcactggacccagcctacaccaccctggaatttgagaatgtgcaggtgttgacgatgggcaatgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtgtgagtggccccgacccaggggacaggcagccacccctgagaccccacagccctcaccgccaggtggctcagggtctgagccctataagctcctgccgggagccgtcgccacaatcgtcaagcccctctctgccatcccccagccgaccatcaccaagcaggaagttatctag

SEQ ID NO: 4 - protein, 474 aa, Homo sapiens
MRLSKTLVDMDMADYSAALDPAYTTLEFENVQVLTMGNDTSPSEGTNLNAPNSLGVSALCAICGDRATGKHYGASSCDGCKGFFRRSVRKNHMYSCRFSRQCVVDKDKRNQCRYCRLKKCFRAGMKKEAVQNERDRISTRRSSYEDSSLPSINALLQAEVLSRQITSPVSGINGDIRAKKIASIADVCESMKEQLLVLVEWAKYIPAFCELPLDDQVALLRAHAGEHLLLGATKRSMVFKDVLLLGNDYIVPRHCPELAEMSRVSIRILDELVLPFQELQIDDNEYAYLKAIIFFDPDAKGLSDPGKIKRLRSQVQVSLEDYINDRQYDSRGRFGELLLLLPTLQSITWQMIEQIQFIKLFGMAKIDNLLQEMLLGGSPSDAPHAHHPLHPHLMQEHMGTNVIVANTMPTHLSNGQMCEWPRPRGQAATPETPQPSPPGGSGSEPYKLLPGAVATIVKPLSAIPQPTITKQEVI

SEQ ID NO: 5 - DNA, 24 bp, Artificial Sequence
  Synthetic primer HNF-1S
atggtttctaaactgagccagctg

SEQ ID NO: 6 - DNA, 22 bp, Artificial Sequence
  Synthetic primer HNF-1A
ttactgggaggaagaggccatc

SEQ ID NO: 7 - DNA, 24 bp, Artificial Sequence
  Synthetic primer HNF-4A2S
atgcgactctccaaaaccctcgtc

SEQ ID NO: 8 - DNA, 25 bp, Artificial Sequence
  Synthetic primer HNF-4A2A
ctagataacttcctgcttggtgatg

SEQ ID NO: 9 - DNA, 18 bp, Artificial Sequence
  Synthetic primer HNF-1-598S
cggaggaaccgtttcaag

(19) United States
(12) Patent Application Publication
(10) Pub. No.: US 2007/0105224 A1
(43) Pub. Date: May 10, 2007

(54) Genetic modification of liver cells to enhance metabolic and physiological efficacy

(75) Inventors: Chin-Yu Lin, Chiayi County (TW); See-Chang Huang, Taipei City (TW); Chun-Min Liu, Changhua County (TW)

Correspondence Address: BIRCH STEWART KOLASCH & BIRCH, PO BOX 747, FALLS CHURCH, VA, 22040-0747, US

(73) Assignee: Industrial Technology Research Institute

(21) Appl. No.: 11/267,419

(22) Filed: Nov. 7, 2005

(51) Int. Cl. C12N 15/867; C12N 5/08

(52) U.S. Cl. 435/456; 435/370; 435/325

(57) ABSTRACT

Retroviral vectors containing hepatic nuclear factor 1 or hepatic nuclear factor 4α, and cells comprising the same are provided.

### BACKGROUND

The invention relates to molecular biology and microbiology, and more particularly, to genetic modification of liver cells.

Bioartificial liver (BAL) using liver cells as a key element is one of the therapeutics approved by the Food and Drug Administration (FDA) and may serve as a temporary liver support for patients with acute liver failure, keeping them alive until their own organ can recover or until a suitable organ becomes available for transplantation. At least eight BAL developing companies are presently conducting clinical trials, however, BAL technology is restricted by the requirement of a stable liver cell line that provides the functions of an intact liver. Since human primary liver cells cannot be easily obtained, only VitaGen incorporated utilizes C3A, human immortalized hepatic cells, as their cell source and the rest of the BAL-developing companies uses primary liver cells derived from pig. Porcine hepatic cells are easily obtainable and similar to human liver cells in physiological and metabolic functions, however, interspecies transmission of pathogens and immunological rejection are of concerns. In addition, human liver cells such as C3A are relatively easily obtainable, but with poor physiological and metabolic functions compared to primary liver cells. It is, therefore, a desperate need to provide a stable and functional cell source for artificial liver.

### SUMMARY

Liver cells cultured in vitro usually lose important hepatic functions including physiological and metabolic functions. It was found that most important hepatic functions are regulated by hepatic nuclear factor (HNF) which is low expressed in liver cells cultured in vitro. In particularly, HNF-1 and HNF-4α are highly expressed in well differentiated liver cells. Genetic modification of liver cells for the improvement of HNF-1 and HNF-4α expression was expected to enhance physiological and metabolic efficacy of liver cells. The inventors cloned human HNF-1 and HNF-4α cDNA, introduced them into murine stem cell virus (MSCV) as a vector, and transiently expressed them in liver cell lines including HepG2-C3A (C3A), THLE-2, and Huh 7. The results show that the expression of genes including CYP1A2, CYP3A4, UGT1A1, PCK1, GLUD1, DGAT1, and CPS1 in the transfected liver cells were times that of the original cells. Stably expressed cell line was screened by G418 and the results are consistent to the transiently expressed one. The invention was then achieved.

In one aspect of the invention, a retroviral expression vector is provided. The retroviral expression vector includes a hepatic nuclear factor 1 (HNF-1) gene composed of a nucleotide sequence of SEQ ID NO: 1.

A cell comprising the above mentioned retroviral expression vector is also provided. One embodiment of the cell is a hepatic cell, such as HepG2-C3A, THLE-2, and Huh7. Genes in the cell may be highly expressed, such as CYP1A2 (cytochrome P450 subfamily), CYP3A4 (cytochrome P450 subfamily), UGT1A1 (UDP glycosyltransferase 1 family, polypeptide A1), PCK1 (phosphoenolpyruvate carboxykinase 1), GLUD1 (L-glutamate dehydrogenase), DGAT1 (diacylglycerol O-acyltransferase), ALB(albumin), F9, or CPS1 (carbamoyl phosphate synthetase I).

In another aspect of the invention, a retroviral expression vector is provided, which includes a hepatic nuclear factor 4α (HNF-4α) gene composed of a nucleotide sequence of SEQ ID NO: 2.

In addition, a cell comprising the above mentioned retroviral expression vector is provided. One embodiment of the cell is a hepatic cell, such as HepG2-C3A, THLE-2, or Huh7. Genes in the cell may be highly expressed, such as CYP1A2 (cytochrome P450 subfamily), CYP3A4 (cytochrome P450 subfamily), UGT1A1 (UDP glycosyltransferase 1 family, polypeptide A1), PCK1 (phosphoenolpyruvate carboxykinase 1), GLUD1 (L-glutamate dehydrogenase), DGAT1 (diacylglycerol O-acyltransferase), ALB(albumin), F9, or CPS1 (carbamoyl phosphate synthetase I).

### BRIEF DESCRIPTION OF THE DRAWINGS

The present invention will be more fully understood and further advantages will become apparent when reference is made to the following description of the invention and the accompanying drawings in which:

FIG. 1 illustrates electrophoresis photographs of PCR products of HNF-1 (A) and HNF-4α (B). The two products were then respectively cloned into a retroviral expression vector, pMSCVneo (BD™ Biosciences Clontech).

FIG. 2 illustrates transient expression of HNF-1/pMSCV in THLE-2 hepatic cell lines. The expression of albumin mRNA was elevated.

FIG. 3A and 3B illustrate transient expression of HNF-1/pMSCV and HNF-4α/pMSCV in Huh-7 (FIG. 3A) and C3A (FIG. 3B) hepatic cell lines. The expression of UGT1A1 (UDP glycosyltransferase 1 family, polypeptide A1) mRNA was elevated.

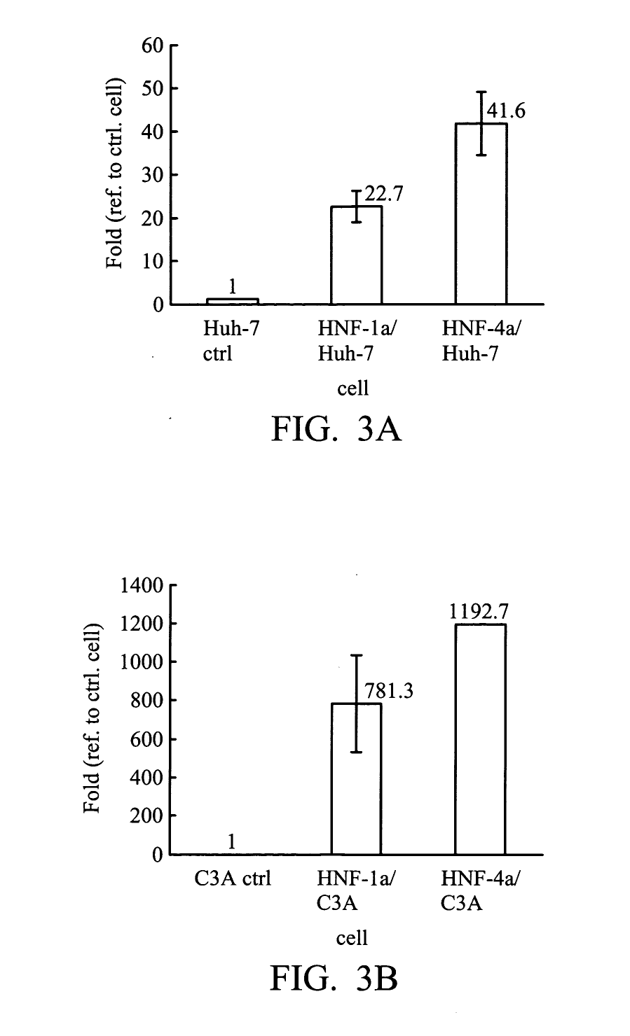

FIG. 4 illustrates transient expression of HNF-1/pMSCV and HNF-4α/pMSCV in Huh-7 and C3A hepatic cell lines. The expression of CYP3A4 (cytochrome P450 subfamily) mRNA was elevated.

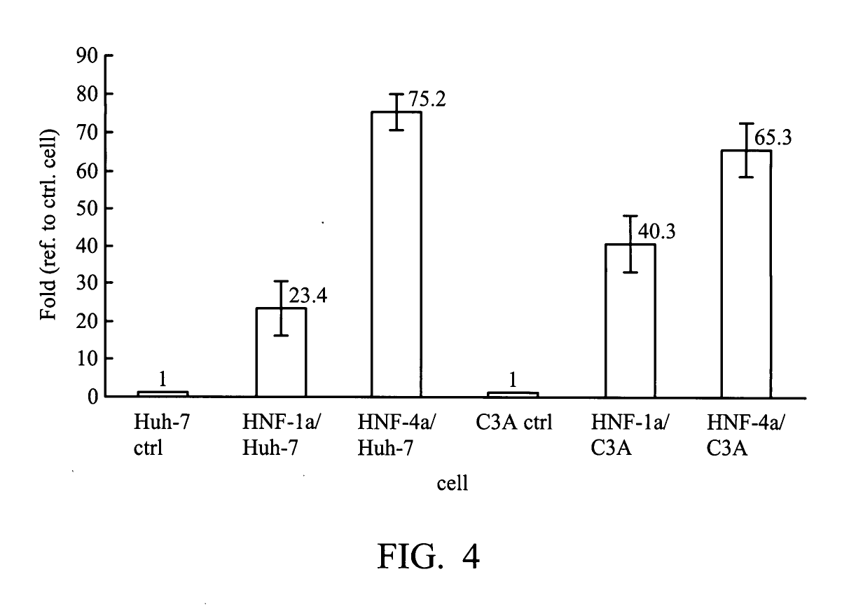

FIG. 5 illustrates Q-PCR results of albumin expression in HNF-1 and HNF-4α stably expressed C3A cell lines.

FIG. 6 illustrates Q-PCR results of UGT1A1 (UDP glycosyltransferase 1 family, polypeptide Al) expression in HNF-1 and HNF-4α stably expressed C3A cell lines.

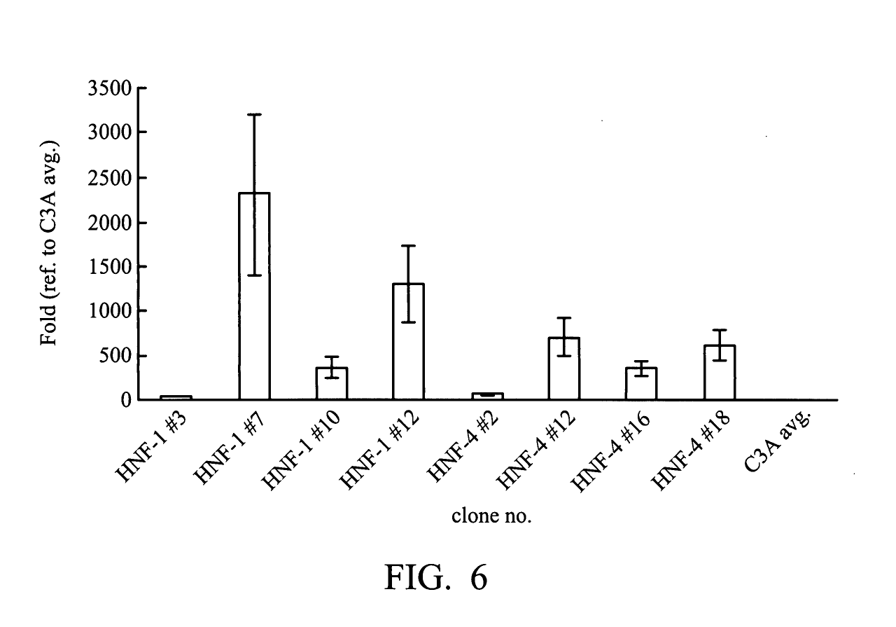

FIG. 7 illustrates Q-PCR results of F9 expression in HNF-1 and HNF-4α stably expressed C3A cell lines.

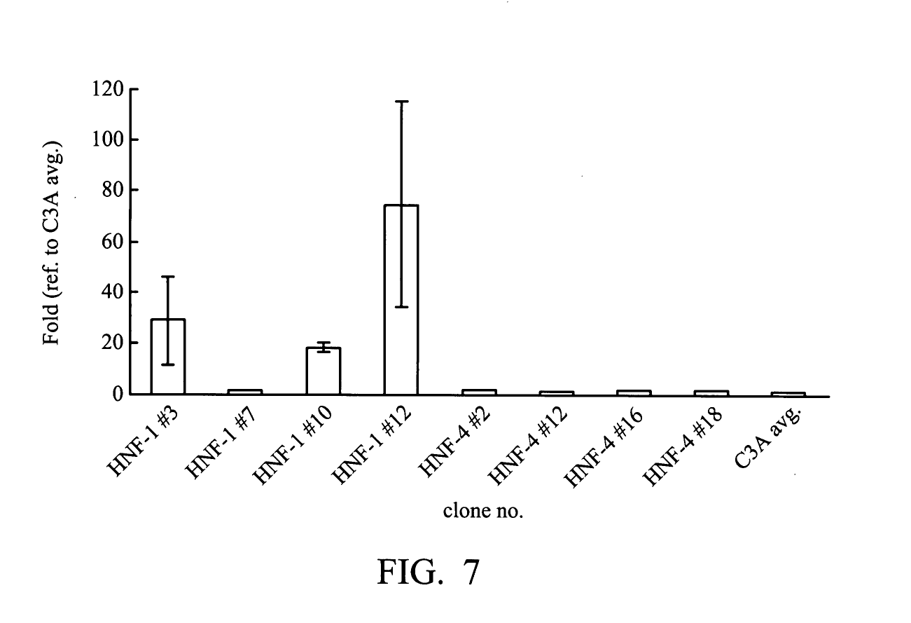

FIG. 8 illustrates Q-PCR results of PCK1 (phosphoenolpyruvate carboxykinase 1) expression in HNF-1 and HNF-4α stably expressed C3A cell lines.

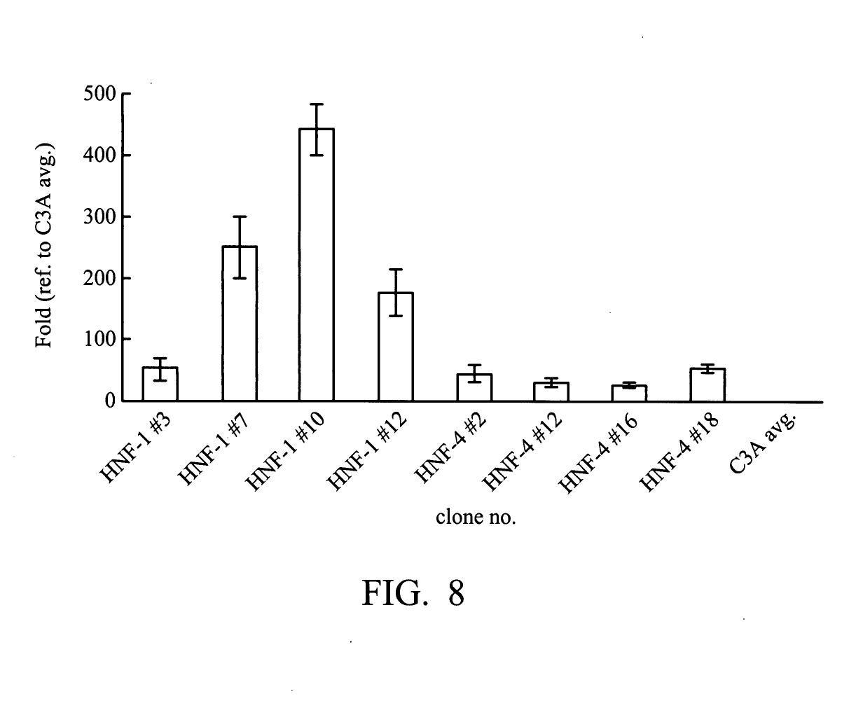

FIG. 9 illustrates Q-PCR results of CPS1 (carbamoyl phosphate synthetase 1) expression in HNF-1 and HNF-4α stably expressed C3A cell lines.

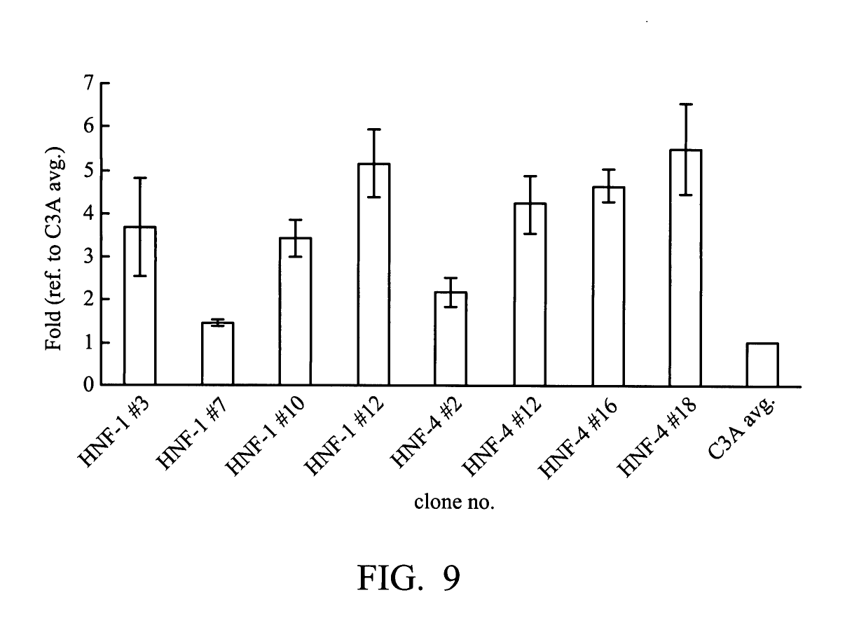

### DETAILED DESCRIPTION

Retroviral vectors containing HNF-1 or HNF-4α CDS, and cells comprising the same are provided.

The inventors intended to modify liver cells to enhance metabolic and physiological efficacy thereof. With a thoroughly search of the related art, the inventors found that well differentiated liver cells with intact hepatic functions feature two highly expressed genes, hepatic nuclear factor 1 (HNF-1) and hepatic nuclear factor 4α (HNF-4α). For example, WO04016813A2 discloses diagnostic methods and therapeutics for liver or colon cancer involving hepatocyte nuclear factor 1α (HNF-1α), a tumor suppressor gene. WO05000335A2 discloses diagnosis and treatment methods related to aging, especially of liver. In this study, mouse genes differentially expressed in comparisons of older and younger livers by gene chip analysis have been identified, as have corresponding human genes and proteins. HNF gene was one of these genes. WO9811254 discloses that the analysis of mutations in the HNF-1α, HNF-1β, and HNF-4α genes can be diagnostic for diabetes.

In addition, a study for the establishment of highly functional liver cells by transfecting HNF-4 gene for the development of BAL was disclosed in Cell Transplant. 13(4):393-403, 2004. An adenoviral vector carrying rat HNF-4 cDNA was transfected to hepatoma-derived cell lines, HepG2 and Huh-7. Expression of liver-specific genes in cells infected by the adenovirus vector expressing HNF-4, such as apolipoproteins, alpha1-antitrypsin (alpha1-AT), phosphoenolpyruvate carboxy-kinase, cytochrome P450 families, and glutamine synthetase was analyzed. It was found that cells overexpressing HNF-4 removed ammonia from medium supplemented with NH₄Cl to a greater extent than control cells. These findings demonstrated that transfected cell lines restored differentiated gene expressions and liver-specific functions by the overproduction of HNF-4. However, it is to be noted that rat HNF-4 was applied rather than human hepatocyte nuclear factors. In addition, this study only has a transient transfection rather than a stable expression system, and a long term expression profile cannot be expected. Moreover, albumin expression was not elevated in this study.

A medium was developed for the improvement of C3A cell metabolism in Journal of Hepatology 41:599-605, 2004. The improvement of C3A cell metabolism is about 2-5 times original C3A cell line.

A highly differentiated human hepatoma cell line BC2 is disclosed in Eur. J. Biochem. 268: 1448-1459, 2001 discloses. BC2 cells express the most relevant cytochrome P-450 (CYP) isozyme activities (CYP1A1/2, 2A6, 2B6, 2C9, 2E1, and 3A4) and conjugating enzymes (glutathione S-transferase and UDP-glucuronyltransferase) and also respond to model inducers. Methylcholanthrene induced an increase in CYP1A1/2 enzyme activity (8-fold), phenobarbital induced CYP2B6 activity (1.7-fold), and dexamethasone induced CYP3A4 activity (5-fold). In addition, mRNA of HNF-4, HNF-1, C/EBP-a, and C/EBP-b genes is highly expressed in BC2 cell.

It was found that hepatocyte nuclear factor 4α enhances the hepatocyte nuclear factor 1α-mediated activation of transcription in Nucleic Acids Research 32(8):2586-2593, 2004. HNF-4α enhances the expression of HNF-1α by promoter binding and protein-protein interaction, and the expression of HNF-4α can be enhances by HNF-1α through promoter binding and inhibited through protein-protein interaction.

Treatment of FHF (fulminant hepatic failure) rats with a bioartifical liver (BAL) untilizing isolated functional hepatocytes is disclosed in Journal of Surgical Research 85:243-250, 1999. BAL treatment resulted in an increased DNA binding of transcription factors engaged in the modulation of hepatocyte proliferation and liver-specific gene expression. It is also found that intrasplenic hepatocyte transplantation prolonged survival in FHF rats and triggered hepatocyte proliferation in the native liver. The latter effect was associated with accelerated expression of HGF and c-met mRNA in the liver and lowering of blood HGF and TGF-β1 levels. The factors such as STAT3, HNF-1, HNF-4, or C/EBP were also altered.

A study for the establishment of a CYP3A4 inducible model for in vitro analysis of human drug metabolism using a BAL is disclosed in Hepatology 37:665-673, 2003. The BAL is composed of the functional hepatocellular carcinoma cell (HCC) line FLC-5. A radial-flow bioreactor (RFB), which is a carrier-filled type bioreactor, was used for 3-dimensional perfusion culture of FLC-5 cells. The CYP3A4 mRNA expression level 48 hours after rifampicin treatment in the RFB was approximately 100 times higher than that in a monolayer culture.

From these references, it is found that HNF-1 and HNF-4α are highly expressed in well differentiated liver cells with intact functions, but low expressed or not expressed in those undifferentiated or dedifferentiated cancerous liver cells or stem cells which has low metabolic or physiological efficacy. The inventors utilized expression vectors containing human HNF-1 or HNF-4α genes for the transfection of C3A hepatic cells. Stably expressed cell lines were then established. The detoxification, metabolism and physiological functions related genes of the stably expressed cell lines were analyzed by real time PCR. It was found that the stably expressed cell lines have more mRNA expression in detoxification, metabolism, and physiological related genes. In addition, the cells can be easily obtained and cultured with low cost.

It is, therefore, provided a retroviral expression vector. The retroviral expression vector includes a hepatic nuclear factor 1 (HNF-1) gene composed of a nucleotide sequence of SEQ ID NO: 1. The retroviral expression vector can be a pMSCVneo expression vector (BD™ Biosciences Clontech).

A cell comprising the above mentioned retroviral expression vector is also provided. One embodiment of the cell is a hepatic cell, such as HepG2-C3A, THLE-2, or Huh7. Genes in the transformed cells are comparably higher than the parental cells, such as CYP1A2 (cytochrome P450 subfamily), CYP3A4 (cytochrome P450 subfamily), UGT1A1 (UDP glycosyltransferase 1 family, polypeptide A1), PCK1 (phosphoenolpyruvate carboxykinase 1), GLUD1 (L-glutamate dehydrogenase), DGAT1 (diacylglycerol O-acyltransferase), ALB(albumin), F9, or CPS1 (carbamoyl phosphate synthetase I). The cell can be applied for bioartificial liver.

In addition, a retroviral expression vector including a hepatic nuclear factor 4α (HNF-4α) gene composed of a nucleotide sequence of SEQ ID NO: 3 is provided. The retroviral expression vector can be a pMSCVneo expression vector (BD™ Biosciences Clontech).

Moreover, a cell comprising the above mentioned a retroviral expression vector is provided. The cell can be a hepatic cell, such as HepG2-C3A, THLE-2, or Huh7. Genes in the transformed cells are comparably higher than the parental cells, such as CYP1A2 (cytochrome P450 subfamily), CYP3A4 (cytochrome P450 subfamily), UGT1A1 (UDP glycosyltransferase 1 family, polypeptide A1), PCK1 (phosphoenolpyruvate carboxykinase 1), GLUD1 (L-glutamate dehydrogenase), DGAT1 (diacylglycerol O-acyltransferase), ALB(albumin), F9, or CPS1 (carbamoyl phosphate synthetase I). The cell can be, therefore, applied for bioartificial liver.

Hepatitis virus infects well differentiated liver cells with intact functions. The HNF-1 or HNF-4α stably expressed cells are expected to be used as a host for hepatitis virus. The susceptibility of the HNF-1 or HNF-4α stably expressed C3A to hepatitis virus can be tested under the stimulation of a known inducer such as DMSO or Cortisone. The HNF-1 or HNF-4α stably expressed cells may be a platform for the screening of drugs against hepatitis virus. The HNF-1 or HNF-4α stably expressed cells may be provided for ex vivo infection and replication of hepatitis virus.

The key for the differences among stem cells, hepatitis virus-infected cells, cancerous hepatic cells, and normal hepatic calls is the level of differentiation. The embodiments of the retroviral vector can be transfected into murine stem cells for the induction of differentiation and an ex vivo differentiation technology can then be established. The differentiated cells must have basic metabolism, detoxification, and physiological functions of a hepatic cell.

The advantages of the invention include:

1. Human cells were transfected with human genes. No cross-species problems exist.

2. HNF-1 has better induction effect than HNF-4α in the enhancement of the metabolism in liver cells as exhibited in the stable clones of the invention.

3. The stable C3A clones screened in the invention exhibit highly expressed metabolic function with at least five-week passages.

4. Liver cell lines induced by HNF-4α or HNF-1 have at least 4-fold elevation of albumin gene expression than the parental cells.

Practical examples are described herein.

### EXAMPLES

### Example 1

### PCR Cloning of HNF-1 and HNF-4α cDNA

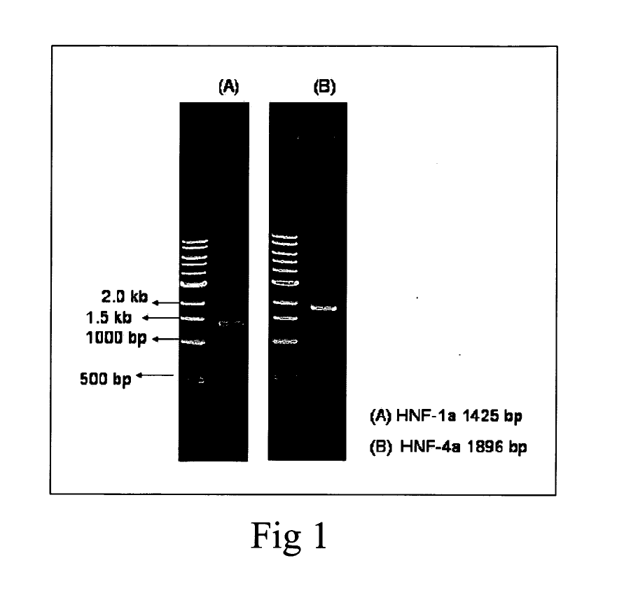

Primers for HNF-1 open reading frame (ORF) were designed as HNF-1S: 5′-ATGGTTTCTAAACTGAGCCAGCTG-3′ (SEQ ID NO: 5) and HNF-1A: 5′-TTACTGGGAGGAAGAGGCC ATC-3′ (SEQ ID NO: 6). Primers for HNF-4α ORF were designed as HNF-4A2S: 5′-ATGCGACTCTCCAAAACCCTC GTC-3′ (SEQ ID NO: 7) and HNF-4A2A: 5′-CTAGATAACTTCCTGCTTGGTGATG-3′ (SEQ ID NO: 8). Sequencing primer HNF-1-598S: 5′-CGGAGGAACCGTTTCAAG-3 (SEQ ID NO: 9) was also designed for the long HNF-1 ORF. PCR was performed by the reaction mixture of: Huh-7 cDNA 60 ng total RNA converted, pfu DNA polymerase 1.25 units, 10 μM primer pairs 2.5 μl, 2.5 mM dNTP 4 μl, and 10×PCR buffer 5 μl with the thermal cycling of ±94° C. 3 min; 94° C. 40 sec, 61° C. 40 sec, 72° C. 3 min 10 cycle; 94° C. 40 sec, 58° C. 40 sec, 72° C. 3 min 25 cycle; 58° C. 40 sec, 72° C. 7 min, 25° C. ∞. The electrophoresis results of the PCR products are shown in FIG. 1. PCR product of HNF-1α coding sequence (CDS) is 1896 bp and that of HNF-4α CDS is 1425 bp. HNF-1 and HNF-4α CDS were then cloned into pGEM®-T Easy vector (Promega™) and sequenced by HNF-1-598S, T7 and SP6 primers. Results confirmed the sequences of HNF-1/pGEM-T clone 2 and HNF-4α/pGEM-T clone 2.

### Example 2

### Construction of Expression Vector of HNF-1 and HNF-4α

HNF-1/pGEM-T clone 2 and HNF-4α/pGEM-T clone 2 were digested by restricted enzymes EcoRI and HpaI separately and HNF-1 and HNF-4α CDS were recovered. The two fragments were cloned into pMSCVneo vector (Clontech™) respectively and the transformants were transfected into eukaryotic cells for the expression of the cloned genes. The clones, HNF-1/pMSCV clone 3 and HNF-4α/pMSCV clone 3, were confirmed by MSCV5′ and MSCV3′ primers.

### Example 3

### Establishment of Stable Transfectant with HNF-1/pMSCV and HNF-4α/pMSCV Retroviral Vectors

1.6 μg plasmid DNA of HNF-1/pMSCV and HNF-4α/pMSCV were mixed with 4 μl of lipofectamine2000® and the volume was adjusted to 200 μl with OPTI-MEM® and the reaction was stayed at RT for 30 min. PT67 package cells were cultured with 800 μl culture media in a 6-well culture dish 16 hours prior to transfection. The transfection mixture was added into the 6-well culture dish and culture was continued for 24 hours. The culture was continued after refreshing the culture media and 400 μg/ml G418 antibiotics was added to select the cells. After 10˜15 day passages and G418 selection, stably expressed clones, PT67 cells containing HNF-1/pMSCV or HNF-4α/pMSCV retroviral vectors were obtained. Retroviral titer was determined as ˜10⁵ cfu/ml (BDTM Biosciences Clontech protocol no.PT3132-1) which is sufficient for eukaryotic transfection experiments.

### Example 4

### Transient Transfection of HNF-1/pMSCV and HNF-4α/pMSCV in Liver Cells

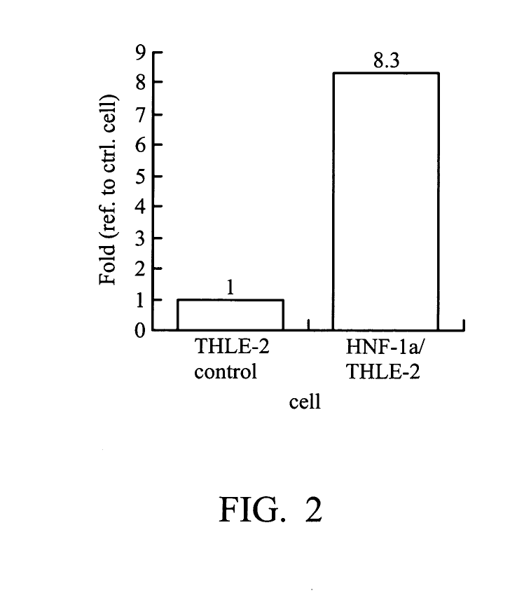

PT67 cell line in which the above mentioned retroviral vectors are stably expressed were cultured to the confluence of 50% and seeded into a 10 cm culture dish. Three ml non-G418 culture medium was added and the cell culture was collected after 24 hour culturing. The cell culture was filtrated by 0.45 μM filter and the retroviral transfection solution was obtained. Liver cell lines C3A, Huh-7, and THLE-2 were respectively seeded in a 6-well culture dish 12 hours prior to transfection. The culture medium was discharged and 1 ml of the retroviral transfection solution was added into the 6-well culture dish for 48-hour transfection. Transfected cells were harvested and total RNA was extracted for cDNA synthesis and real time PCR. Real time PCR was performed by the reaction mixture of: cDNA 66 ng total RNA converted, 10 μM primer pairs 0.4 μl, 2×SYBR Green® PCR Master Mix with thermal cycling of: 50° C. 2 min; 95° C. 10 min; 95° C. 15 sec, 60° C. 1 min 40 cycles. Results are shown in FIG. 2˜4.

### Example 5

### Establishment of HNF-1 and HNF-4α Stably Expressed C3A Liver Cell Line

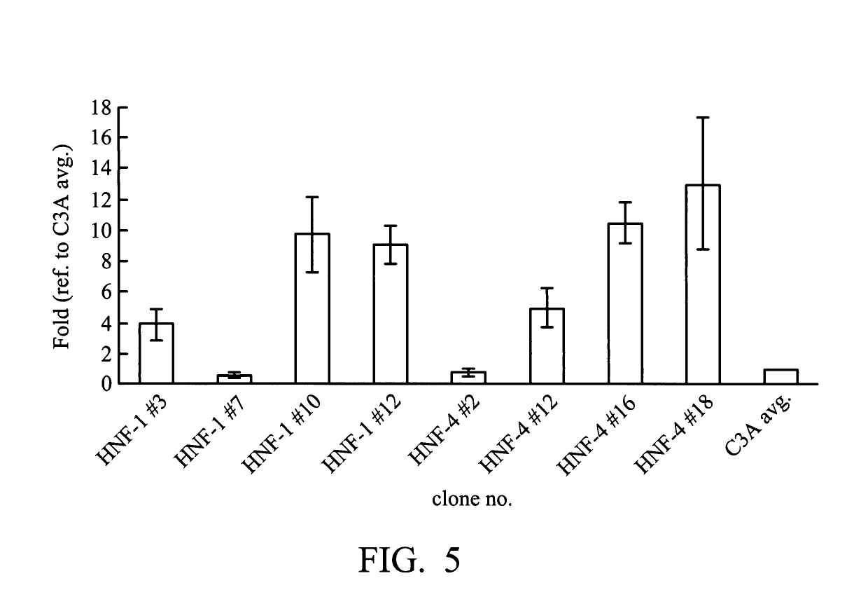

Retroviral transfection was performed as above described. After transfection, the transfected C3A cell line cultured in a 6-well dish was selected by G418. With serial dilution, single cell was cultured in a 96-well dish. Stable trasfectants of HNF-1/pMSCV and HNF-4α/pMSCV were obtained 5 weeks after transfection. Transfectants were harvested and total RNA was extracted for cDNA synthesis and real time PCR. Results are shown in FIG. 5˜11. The tested genes and their expression improvement are listed in table 1.

TABLE 1

| | Expression | | |
|---|---|---|---|
| genes | improvement | | Function |
| Albumin (ALB) | 4-12 | folds | |
| Carbamoyl phosphate | 1.5˜5 | folds | Rate-limiting enzyme that |
| synthetase 1 (CPS1) | | | catalyzes the first step |
| | | | of the hepatic urea cycle |
| Cytochrome P450, | 10˜90 | folds | |
| family 1, subfamily | | | |
| A, polypeptide 2 | | | |
| (CYP1A2) | | | |
| Cytochrome P450, | 200˜1000 | folds | P450 protein are |
| family 3, subfamily | | | monooxygenases which |
| A, polypeptide 4 | | | catalyze many reactions |
| (CYP3A4) | | | involved in drug |
| | | | metabolism and synthesis |
| | | | of cholesterol, steroids, |
| | | | and other lipids |
| Diacylglycerol | 80˜180 | folds | The enzyme encoded by |
| acytransferase 1 | | | this gene utilizes |
| (DGAT1) | | | diacylglycerol and fatty |
| | | | acyl CoA as substrates in |
| | | | order to catalyze the |
| | | | final stage of |
| | | | triacylglycerol synthesis |
| Coagulation factor | 20˜70 | folds | |
| IX (F9) | | | |
| Glutamate | 10˜30 | folds | GLUD1 has a central role |
| dehydrogenase 1 | | | in nitrogen metabolism |
| (GLUD1) | | | and ammonia |
| | | | detoxification |
| Hepatocyte nuclear | 400˜800 | folds | |
| factor 1α (HNF-1α) | | | |
| Hepatocyte nuclear | 200˜800 | folds | |
| factor 4α (HNF-4α) | | | |
| Phosphoenolpyruvate | 50˜400 | folds | This gene is a main |
| carboxykinase 1 | | | control point for the |
| (PCK1) | | | regulation of |
| | | | gluconeogenesis |
| UDP | 100˜2000 | folds | This gene encodes a UDP- |
| glycosyltransferase | | | glucuronosyltransferase, |
| 1 faminly | | | an enzyme of the |
| polypeptide A1 | | | glucuronidation pathway |
| (UGT1A1) | | | that transforms small |
| | | | lipophilic molecules, |
| | | | such as steroids, |
| | | | bilirubin, hormones, and |
| | | | drugs, into water- |
| | | | soluble, excretable |
| | | | metabolites |

While the invention has been described by way of example and in terms of preferred embodiment, it is to be understood that the invention is not limited thereto.

What is claimed is:

1. A retroviral expression vector, comprising a hepatic nuclear factor 1 (HNF-1) gene composed of a nucleotide sequence of SEQ ID NO: 1.

2. The retroviral expression vector as claimed in claim 1, wherein the retroviral expression vector is a pMSCVneo expression vector (BD™ Biosciences Clontech).

3. The retroviral expression vector as claimed in claim 1, which is deposited in

4. A cell comprising a retroviral expression vector as claimed in claim 1.

5. The cell as claimed in claim 4, which is a hepatic cell.

6. The cell as claimed in claim 5, which is HepG2-C3A, THLE-2, or Huh7.

7. The cell as claimed in claim 4, wherein the cell has higher expressed genes comparing to the parental cell, consisting of CYP1A2 (cytochrome P450 subfamily), CYP3A4 (cytochrome P450 subfamily), UGT1A1 (UDP glycosyltransferase 1 family, polypeptide A1), PCK1 (phosphoenolpyruvate carboxykinase 1), GLUD1 (L-glutamate dehydrogenase), DGAT1 (diacylglycerol O-acyltransferase), ALB(albumin), F9, and CPS1 (carbamoyl phosphate synthetase I).

8. The cell as claimed in claim 4, which is for bioartificial liver.

9. A retroviral expression vector, comprising a hepatic nuclear factor 4α (HNF-4α) gene composed of a nucleotide sequence of SEQ ID NO: 3.

10. The retroviral expression vector as claimed in claim 9, wherein the retroviral expression vector is a pMSCVneo expression vector (BD™ Biosciences Clontech).

11. The retroviral expression vector as claimed in claim 9, which is deposited in

12. A cell comprising a retroviral expression vector as claimed in claim 9.

13. The cell as claimed in claim 12, which is a hepatic cell.

14. The cell as claimed in claim 13, which is HepG2-C3A, THLE-2, or Huh7.

15. The cell as claimed in claim 12, wherein the cell has higher expressed genes comparing to the parental cell, consisting of CYP1A2 (cytochrome P450 subfamily), CYP3A4 (cytochrome P450 subfamily), UGT1A1 (UDP glycosyltransferase 1 family, polypeptide A1), PCK1 (phosphoenolpyruvate carboxykinase 1), GLUD1 (L-glutamate dehydrogenase), DGAT1 (diacylglycerol O-acyltransferase), ALB(albumin), F9, and CPS1 (carbamoyl phosphate synthetase I).

16. The cell as claimed in claim 12, which is for bioartificial liver.
